(19) United States
(12) Patent Application Publication
(10) Pub. No.: US 2015/0174354 A1
(43) Pub. Date: Jun. 25, 2015

(54) RESPIRATORY INTERFACE DEVICE FOR DELIVERING GAS TO A USER

(71) Applicant: KONINKLIJKE PHILIPS N.V., EINDHOVEN (NL)

(72) Inventors: Jerome Matula, JR., Apollo, PA (US); Sander Theodoor Pastoor, Utrecht (NL); Peter Chi Fai Ho, Pittsburgh, PA (US); Christoph Dobrusskin, Eindhoven (NL)

(21) Appl. No.: 14/403,583

(22) PCT Filed: May 31, 2013
(86) PCT No.: PCT/IB2013/054501

§ 371 Date: Nov. 25, 2014

Related U.S. Application Data
(60) Provisional application No. 61/654,147, filed on Jun. 1, 2012.

(51) Int. Cl. A61M 16/06; A61M 16/08; A61M 16/00

(52) U.S. Cl. CPC A61M 16/0622; A61M 16/0066; A61M 16/0683; A61M 16/08

(57) ABSTRACT

The present invention relates to a respiratory interface device for delivering a gas to a user (50), comprising: —a connection interface (24) for connecting a hose to deliver gas to the user (50), —a cushion (12) that is adapted to contact a face of the user (50) and to surround at least a part of a nose and/or a mouth of the user (50), and—an inflatable support structure (14) for holding the cushion (12).

### FIELD OF THE INVENTION

The present invention relates to a respiratory interface device for delivering a gas to a user. The present invention further relates to an inflatable support structure for use in such a respiratory interface device.

### BACKGROUND OF THE INVENTION

More and more patients suffer from obstructive sleep apnea or obstructive sleep apnea syndrome (OSA). OSA is usually caused by an obstruction of the upper airway. It is characterized by repetitive pauses in breathing during sleep and is usually associated with a reduction in blood oxygen saturation. These pauses in breathing, called apneas, typically last 20 to 40 seconds. The obstruction of the upper airway is usually caused by reduced muscle tonus of the body that occurs during sleep. The human airway is composed of walls of soft tissue which can collapse and thereby obstruct breathing during sleep. Tongue tissue moves towards the back of the throat during sleep and thereby blocks the air passages. OSA is therefore commonly accompanied with snoring.

Different invasive and non-invasive treatments for OSA are known. One of the most powerful non-invasive treatments is the usage of CPAP (Continuous Positive Airway Pressure) or BiPAP (Bi-Positive Airway Pressure) in which a face mask is attached to a tube and a machine that blows pressurized air into the mask and through the airway in order to keep it open. Positive air pressure is thus provided to a patient through a hose connected to a respiratory interface, such as a face mask, that is worn by the patient. These respiratory interfaces need to closely fit on the patient's face, for instance, to provide an air-tight seal. Usually, the respiratory interface is worn using a headgear with straps that go around the back of the patient's head.

An example of such a CPAP-system is known from WO 2011/022779 A1. The mask or respiratory interface used therein comprises a rigid shell to which the air supplying hose is connected and into which a soft cushion, preferably made of silicone, is integrated or attached to. The silicone cushion builds the mask-to-face interface and contacts the patient's face during use of the mask. If furthermore serves as sealing element for providing an air-tight seal of the mask.

Due to the large rigid mask shells that serve for attaching the air supplying hose and the cushion, such masks have shown to be very bulky. Apart from that, the complex construction of these masks often results in a heavy overall weight of the mask. The rigid mask shell, the seal cushions, the hose and its elbow connect are all relatively bulky and make the mask heavy to wear. Furthermore, the hose causes additional forces acting on the mask structure. Bearing in mind that such masks are usually worn over night when the patient is asleep, this has shown to be uncomfortable for the patient. The wearing of such masks may therefore be unpleasant and painful for the user. Another disadvantage is the obtrusiveness of today's known masks that impairs the user comfort, especially during sleep. Furthermore, bulky respiratory interfaces are absolutely impractical during travel or transport.

At this point it shall be noted that if the term “mask” is used herein, this shall not be understood as a limitation to a special type of embodiment of a mask rather than referring to respiratory interfaces as described herein in general, where applicable and if not stated otherwise. Furthermore, it shall be noted that the presented respiratory interface device is not limited to the treatment of obstructive sleep apnea.

### SUMMARY OF THE INVENTION

It is an object of the present invention to provide a respiratory interface device for delivering a gas to a user that allows to overcome the above-mentioned disadvantages. In particular, it is an object to provide a respiratory interface device that is less bulky, more lightweight and easier to take along and/or easier to store than currently known respiratory interface devices. Further, it is an object to provide a low cost solution of such a respiratory interface device.

According to the present invention, this problem is solved by a respiratory interface device for delivering gas to a user, comprising:

a connection interface for connecting a hose to deliver gas to the user,

a cushion that is adapted to contact a face of the user and to surround at least a part of a nose and/or a mouth of the user, and

an inflatable support structure for holding the cushion.

According to another aspect of the present invention, the above-mentioned problem is solved by an inflatable support structure for use in such a respiratory interface device, wherein the inflatable support structure is adapted for holding a cushion that is arranged to contact a face of a user and to surround at least a part of a nose and/or a mouth of the user.

The invention therefore overcomes the above-mentioned disadvantages by providing an at least partly inflatable mask. The proposed inflatable support structure is adapted for holding the cushion and for creating a flexible, though at least partly rigid structure when being inflated. The inflatable support structure may have various shapes. It provides mechanical stability to the respiratory interface device (mask) and its parts. According to an embodiment, the connection interface for connecting the gas delivery hose may be arranged on or integrated into the inflatable support structure. The respiratory interface device may comprise one or more cushions that may be attached to the inflatable support structure. In other words, the inflatable support structure may include the one or more cushions, however, the inflatable support structure and the one or more cushions may also be realized as separate parts. According to an embodiment the respiratory interface device, in particular the cushion and the inflatable support structure are made of only one material. This embodiment serves for a very low-cost solution. However, it is also conceivable to use different kinds of materials for the cushion and the inflatable support structure as well as for the remaining parts of the respiratory interface device.

An inflatable support structure as proposed according to the present invention mainly has the advantage that it results in a very lightweight overall construction of the mask. Furthermore, since the support structure is in- and/or deflatable, it may, for example, be flat-packed for shipping and only inflated before or during use. When being inflated with a gas, preferably with air, the gas creates enough pressure allowing to stabilize the structure, thereby providing a mechanically stable holding structure for the one or more cushions.

The inflatable support structure preferably comprises one or more chambers that may be filled with gas in order to inflate the support structure. The support structure itself preferably serves as mask shell for holding the one or more cushions when being inflated. The inflation of the support structure may be done by an additional pump and/or by the available pressure of the gas flower that is connected to the connection interface, or by switching the gas blower from gas-to-patient delivery mode to a mask inflation mode. It is to be noted that the present invention should cover all types of masks which comprise an inflatable support structure and a cushion as explained, independent if it is designed as an oral mask, a nasal mask, a full face mask, a total face mask, an alternative mask/nasal pillows.

According to a further embodiment, the cushion is releasably connected to the inflatable support structure. Different kinds of mechanical and other connections are possible to connect the one or more cushions to the inflatable support structure. The one or more cushions may, for example, be clipped into the inflatable support structure. However, also other kinds of connections are possible, e.g. using magnets. A releasable connection between the one or more cushions and the inflatable support structure mainly has the advantage of enabling a fast replacement of the one or more cushions. The one or more cushions may, for example, also be released from the inflatable support structure in order to clean the one or more cushions and/or the inflatable support structure. The respiratory interface device is therefore easy to handle during cleaning. Especially the one or more cushions, which usually build the mask-to-face interface need to be cleaned or washed quite often.

However, the one or more cushions may also be fixed permanently to the inflatable support structure. According to an embodiment of the present invention, the cushion is embedded into the inflatable support structure. This mainly has the advantage of an increased stability of the connection between the cushion and the inflatable support structure. An unintentional release of the cushion from the inflatable support structure (from the mask shell) is thereby effectively prevented. The one or more cushions may, for example, be integrated or embedded into a cavity that is formed-in the inflatable support structure. The inflatable support structure and/or the one or more cushions are arranged to provide room for a cavity for receiving the nose and/or the mouth of the patient. This cavity is created when the inflatable support structure is inflated. On the other side the connection interface is preferably connected to this cavity, so that the pressurized gas (e.g. pressurized air) is delivered to the patient through the described mouth- and/or nose-receiving cavity. Preferably, the one or more cushions and the inflatable support structure together form a nasal mask, a mouth mask, a full-face mask or a total-face mask.

According to an embodiment, the one or more cushions and/or the support structure is in the inflated condition pre-shaped and adapted to contours of the face of the patient. The cushion and/or the support structure may, for example, be individually manufactured and adapted to the individual contours of the patient's face. This allows an almost perfect fit, thereby increasing the user's comfort and facilitating the reduction of unwanted air leaks that harm the operation of the mask. However, it is to be noted that this feature is not a mandatory feature, but rather an optional feature. Low cost solutions may have standardized shapes and sizes of cushion and/or support structure.

According to a further embodiment, the inflatable support structure comprises at least one inflatable rib that is adapted to form a frame for holding the cushion and for providing mechanical stability when the at least one rib is inflated. Preferably, the inflatable support structure comprises two or more, or a plurality of inflatable ribs. The inflatable support structure may also consist of one or more inflatable ribs. Such a rib-like structure may, when being inflated, resemble a rigid mask shell made of plastic. A plurality of ribs may be connected to each other and form a skeleton for holding the one or more cushions when the rib-like structure is inflated. The inflatable ribs mainly serve for mechanical stability and create a quasi semi-rigid mask shell. Depending on the specific embodiment, each rib of the rib-like structure may be separately inflatable or uniformly inflatable together with the remaining ribs of the rib-like structure.

According to a further embodiment, the proposed respiratory interface device comprises a rigid frame for providing mechanical stability to the respiratory interface device and for holding the inflatable support structure and/or the cushion.

In this embodiment, the mask shell may be supported by an additional rigid frame. In this case, only parts of the mask, i.e. the inflatable support structure, are inflatable while a rigid base structure or frame still additionally exists. In order to solve the above-mentioned disadvantages known from the prior art, this rigid frame preferably comprises or consists of a lightweight material. The shape of the rigid frame is preferably adapted to the shape of the inflatable support structure in the inflated condition. In order to save storage room and provide a space-saving construction of the respiratory interface device, the rigid frame may, for example, be foldable from an initially flat sheet. Thus, the rigid frame may be assembled from an initially flat sheet by folding along certain lines and connecting certain edges with each other.

Alternatively, the inflatable support structure may be fixed permanently to the rigid frame or be embedded into the rigid frame. When being inflated, the inflatable support structure then serves as an additional mechanical stability support for the frame enabling a quite stable mask shell construction, which may be deflated when space needs to be saved during use or storage.

According to an embodiment of the present invention, the support structure is inflatable from a deflated to an inflated condition, wherein the support structure has a substantially flat shape in the deflated condition. Preferably, the support structure has a shape of a flat sheet in the deflated condition. In this embodiment, the inflatable support structure together with the cushion may form a very flat package, when the support structure is deflated, i.e. when the respiratory interface device is not in use. Especially in case the cushion is integrated in the inflatable support structure, the mask can be flat-packed for shipping and/or storing. This allows to reduce transport costs and also increases the handling for the user/patient.

According to a further embodiment, the inflatable support structure and/or the cushion is at least partly made of an elastic foil. In an exemplary embodiment, the inflatable support structure and the cushion consist of one material and form an integral common part. An elastic heat sealing foil is preferably used thereto. Compared to rigid foils, which show wrinkling, elastic foils enable a rigid and still smooth surface when being inflated under proper tension. This allows an easy inflation as well as an easy deflation. Such elastic heat sealing foils are furthermore very cheap to produce and cheap to purchase. An inflatable support structure that is made of such an elastic foil may at the same time also serve as cushion for contacting the patient's face during use, when the inflatable support structure is inflated, so that a separate cushion is no longer needed, i.e. the cushion is integrated or embedded into the inflatable support structure. Apart from that, such a one piece solution made of a single material (e.g. an elastic heat sealing foil) furthermore has the advantage that it is comfortable to wear, since forces are equally distributed due to the air cushion that is created when the inflatable support structure is inflated. In another variant the inflatable support structure comprises a skin comfortable layer that contacts the patient's face. during use

According to a further embodiment of the present invention, the cushion is at least partly inflatable. In other words, the cushion is in this embodiment also inflatable similar as the support structure as described above. In this case the support structure and the cushion may either be one integral common part or also be separate parts. An air-filled cushion shows good damping and therefore increases the user comfort. Due to its flexible behaviour an air-filled cushion optimally aligns itself to the contours of the patient's face. Unwanted air leaks at the mask-to-face interface may thus effectively be prevented. However, it is to be noted that the cushion may also comprise a gel or silicone pad as this is commonly used according to the prior art.

According to an embodiment, the respiratory interface device further comprises a gas inlet for inflating the support structure. Such a gas inlet may, for example, be realized by a small size valve. This gas inlet or valve may be connected to a gas or air supplying apparatus to inflate the support structure prior to the use of the mask. In an alternative embodiment, the cushion is connected to the inflatable support structure and inflatable via the gas inlet. In other words, the cushion and the support structure may be inflated together by supplying air through the same gas inlet. In this way, the cushion and the support structure may be inflated in a single work step.

According to a further embodiment of the present invention, an interior part of the support structure and/or the cushion is connected to the connection interface, wherein the connection interface is arranged to inflate the support structure and/or the cushion. In this case, the inflation of the cushion and/or the support structure is combined with the gas or air delivery with which the patient is supplied. A separate gas inlet as explained above is thus not needed. The above-described gas inlet is then combined with the connection interface for connecting a hose to deliver gas to the patient. This reduces the overall number of required parts of the mask, is easier to handle for the user and also reduces production costs. Apart from that, the user does in this case not need to inflate the mask or parts of the mask prior to using it, since this is done in the same step automatically by starting the gas supply.

According to a further embodiment, the respiratory interface device comprises a head gear that is adapted to fixate the respiratory interface device to the patient's head, wherein the head gear is at least partly inflatable. The head gear itself may also be fully inflatable.

It may, for example, also be inflatable through the same gas inlet that is also used to inflate the support structure and/or the cushion. Similar as an inflatable cushion, an inflatable or at least partly inflatable head gear almost perfectly aligns itself with the contours of the patient's head. The rigidity of the head gear may be adapted by adapting the gas pressure within the head gear. The head gear itself may be realized in different ways. It may, for example, comprise partly or fully inflatable straps that are adapted to go around the back of the head. However, also other constructions of the head gear are conceivable.

According to a still further embodiment, the patient interface is adapted to be foldable from an unfolded, substantially flat shape to a folded shape.

In this embodiment, the cushion and/or the support structure and/or the whole respiratory interface device (mask) may be foldable. In addition, each of these parts may be inflatable as explained above. The mask, the cushion and/or the support structure may thus be arranged to be assembled from an initially flat package. The mask, the cushion and/or the support structure may comprise or consist of a disposable material, such as paper or consumer grade plastics or other low cost materials. The patient interface may then be manufactured from a flat sheet of material. By folding along certain lines and connecting certain edges, the patient interface may be assembled from the initially flat sheet. Such an assembly from a flat package allows storage and transport of the respiratory interface device while it is still a flat package. Consequently, compared to bulky and rigid masks as known from the prior art, storage and transport become easier. In order to get the mask ready for use it only needs to be assembled by folding and then partly or fully inflated as explained above.

### BRIEF DESCRIPTION OF THE DRAWINGS

These and other aspects of the invention will be apparent from and elucidated with reference to the embodiment(s) described hereinafter. In the following drawings

FIG. 1 shows an example of a respiratory interface device according to the state of the art;

FIG. 2 schematically illustrates a first embodiment of the respiratory interface device according to the present invention in a top view;

FIG. 3 schematically illustrates the first embodiment of the respiratory interface device of FIG. 2 in a sectional view;

FIG. 4 schematically illustrates a second embodiment of the respiratory interface device according to the present invention in a top view from a first side;

FIG. 5 illustrates the second embodiment of the respiratory interface device shown in FIG. 4 in another top view from another side;

FIG. 6 shows a sectional view of the second embodiment of the respiratory interface device shown in FIGS. 4 and 5 in a deflated condition;

FIG. 7 shows a sectional view of the second embodiment of the respiratory interface device shown in FIGS. 4 and 5 in an inflated condition;

FIG. 8 schematically illustrates a third embodiment of the respiratory interface device according to the present invention in an unfolded condition; and

FIG. 9 schematically shows the third embodiment of the respiratory interface device according to the present invention in a folded, assembled condition.

### DETAILED DESCRIPTION OF THE DRAWINGS

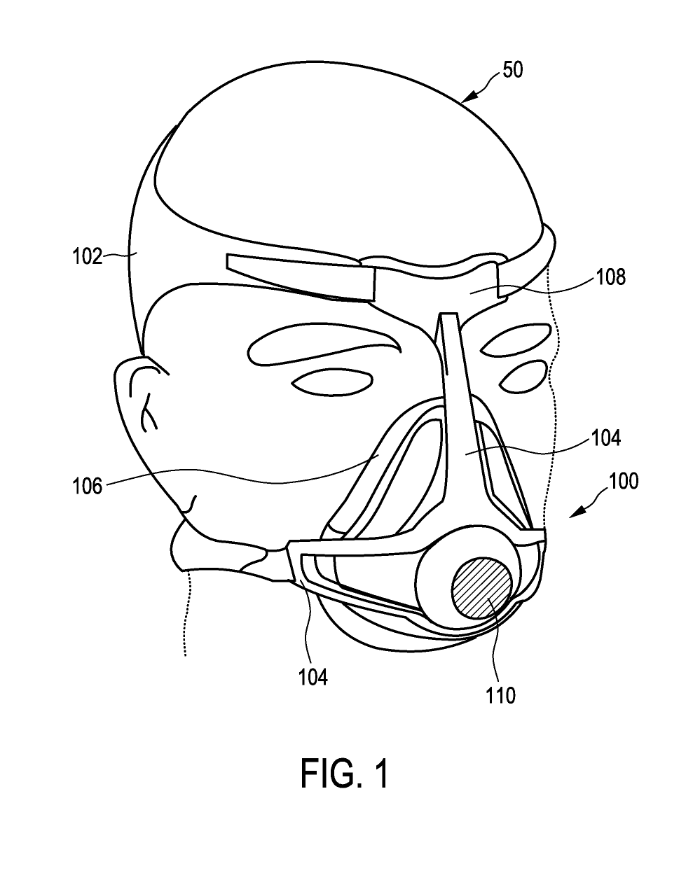

FIG. 1 shows an example of a CPAP-mask according to the prior art. The mask is therein in its entirety denoted with reference numeral 100. The mask, in the following referred to as respiratory interface device 100, is typically used in pressure support systems (CPAP-systems) that supply a flow of gas to the airway of a patient 50. Such respiratory interface devices are well known and are mostly worn on the head using a strap system 102 around the patient's head to hold the mask 100 in place around the airway entry features of the human face. Such masks 100 typically comprise a rigid mask shell 104 to which the head gear 102 is attached. The mask shell 104 is usually made of a rigid material, such as a rigid plastic. The rigid mask shell 104 serves as holding frame for holding a flexible or soft cushion 106. The cushion 106 engages the patient's face when the mask 100 is attached to the patient's face during use. It serves as mask-to-patient interface. These cushions 106 are usually made of silicone or comprise a gel pad in order to establish a soft contact on the patient's face. A further function of these cushions 106 is the sealing of the interior of the mask 100 to the exterior surrounding in order to prevent unwanted air leaks between the mask 100 and the patient's face when pressure is supplied to the patient's airway. The shape of the cushion 106 is thereto preferably adapted to the shape of the user's face.

The illustrated example refers to a so-called full-face mask 100, wherein the cushion 106 surrounds the nose and mouth of the user 50. These full-face masks 100 often comprise an additional cushion support 108 which is usually integrally connected to the mask shell 104, and which is arranged to engage the forehead of the patient 50. The additional cushion support 108 mainly serves to balance the forces that the mask 100 exerts onto the face of the patient 50 and to mechanically stabilize the mask shell 104 and to serve for a correct and comfortable fit of the mask 100. A gas supplying hose (not shown) is usually connected to a connection interface 110 that is preferably attached to or integrated into the rigid mask shell 104.

The disadvantage of today's masks is their obtrusiveness and weight. The rigid mask shell 104, the sealing cushion 106 and the air supplying hose are all relatively bulky and make the mask 100 heavy to wear. Furthermore, the air supplying hose causes additional forces acting on the mask structure, making the mask 100 uncomfortable to wear.

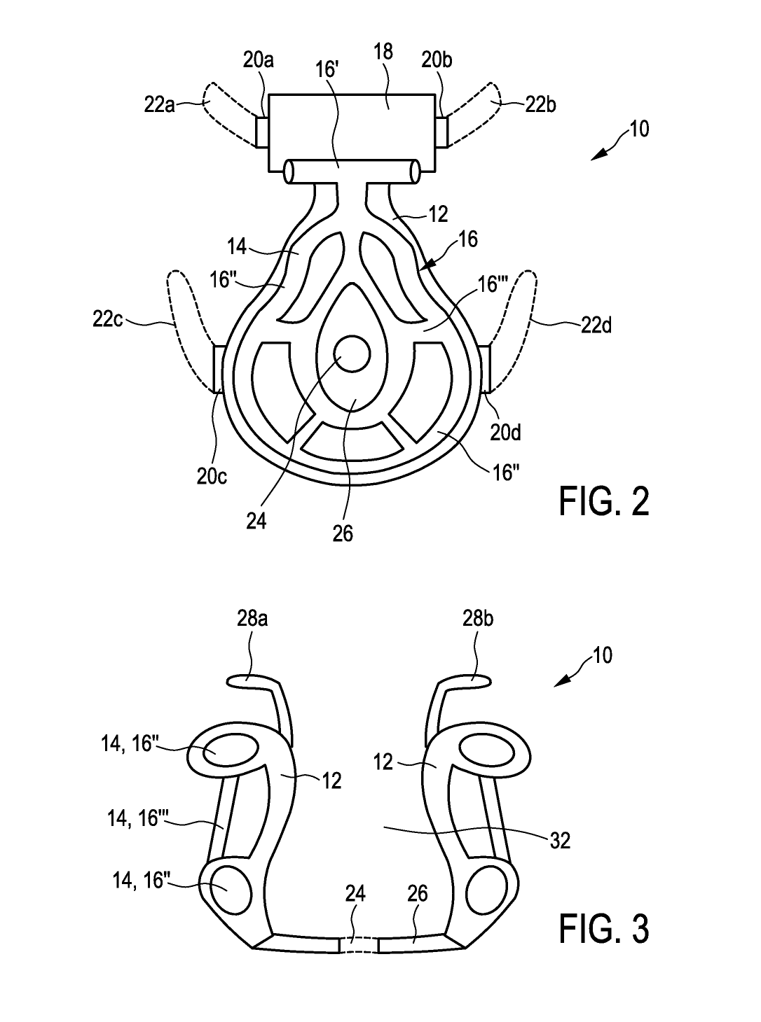

FIG. 2 shows a first embodiment of a respiratory interface device according to the present invention that overcomes the above-mentioned disadvantages of the masks according to the prior art. The respiratory interface device is therein in its entirety denoted with reference numeral 10. The respiratory interface device 10 comprises a cushion 12 and a support structure 14 for holding the cushion 12 and providing mechanical stability to it. The cushion 12 is adapted to contact the face of the user 50 and to surround at least a part of the nose and/or mouth of the user 50, preferably to surround the nose and/or mouth of the user 50. It may, for example, comprise silicone or any other skin comfortable material that provides a sufficient sealing effect. Preferably, the cushion 12 is made of silicone or any other skin comfortable sealing material.

In contrast to the prior art, the support structure 14 for holding the cushion 12 is according to this embodiment inflatable. The inflatable support structure 14 serves as mask shell and replaces the rigid mask shell that is used according to the prior art. The inflatable support structure is realized as an air enforced structure comprising inflatable rips 16 which are adapted to form a mechanically stable frame when the inflatable support structure 14 or the ribs 16, respectively, is/are inflated with air or any other pressurized gas. The support structure 14 is in other words thus inflatable from a deflated to an inflated condition. It preferably has a substantially flat shape in the deflated condition and forms the above-mentioned semi-rigid holding frame when being inflated.

Such an air enforced structure 14 limits the weight by use of less material while maintaining a sufficiently rigid mask support. Due to the very flat shape of the inflatable support structure 14 in the deflated condition, the respiratory interface device 10 is thus easy and space saving to transport or to store.

The cushion 12 may either be releasably connected or fixed permanently to the inflatable support structure 14. A releasable connection between the cushion 12 and the inflatable support structure 14 mainly has the advantage of an easy and fast exchange of the cushion 12 in order to wash or replace the cushion 12 and/or the inflatable support structure 14. However, the inflatable support structure 14 may also be embedded into the cushion 12. In any case, it is preferred that the cushion 12 and the inflatable support structure 14 form a flat package, i.e. have a flat and thin shape, when the inflatable support structure 14 is deflated, i.e. not inflated.

Similar as the respiratory interface devices known from the prior art, the respiratory interface device 10 according to the present invention may also include an additional cushion support 18 which is adapted to engage the patient's forehead and thereby increase the overall stability of the respiratory interface device 10. This additional cushion support 18 may either be a separate part of the cushion 12 or may be integrally connected to the cushion 12. However, it is to be noted that the additional cushion support 18 is not mandatory for the function of the respiratory interface device 10. In case of a provision of such an additional cushion support 18, the inflatable support structure 14 may comprise an additional inflatable rib 16′ which may be either connected to the remaining ribs 16 of the inflatable support structure or may be separately inflatable.

The respiratory interface device 10 furthermore comprises connection member 20a-d that serve for connecting a head gear or head gear straps 22a-d (schematically illustrated with dotted lines) for fixating the respiratory interface device 10 on the patient's head or face. The head gear straps 22a-d may be of various shapes and are not limited to a specific size or shape.

The respiratory interface device 10 according to the present invention further comprises a connection interface 24 for connecting a gas supplying hose to the respiratory interface device 10. This connection interface 24 may either be integrated into or connected to the cushion 12, but may also be integrated or connected to the inflatable support structure 14. In another variant, the connection interface 24 is integrated into or connected to a part of a rigid frame 26 which provides further mechanical stability and serves for a stable and sustainable connection between the gas supplying hose and the respiratory interface device 10. The rigid frame part 26 may, for example, be a rigid or semi-rigid plastic plate. However, it is to be noted that this rigid frame part 26 is only an optional feature and not a mandatory feature of the respiratory interface device 10 according to the present invention. The respiratory interface device 10 may also comprise other rigid frame parts (not shown) for providing mechanical stability to the respiratory interface device 10 and for holding the inflatable support structure 14 and/or the cushion 12.

The connection interface 24 itself may be a simple hole or channel within the mask that may be provided with a thread into which the gas supplying hose may be screwed. However, the gas supplying hose may also be clipped into the connection interface 24 by any clipping mechanism. Other types of mechanical connections are conceivable as well.

Even though FIG. 2 schematically illustrates a full-face mask, this type of mask shall only be an illustrative example. However, the invention shall not be limited to any specific type of mask. It is to be noted that the present invention should cover all types of masks which comprise an inflatable support structure and a cushion as explained, independent if it is designed as an oral mask, a nasal mask, a full-face mask, a total face mask, an alternative mask, or as nasal pillows.

FIG. 3 schematically illustrates a sectional view of the first embodiment of the respiratory interface device 10 shown in FIG. 2. This sectional view visualizes the respiratory interface device 10 in the inflated condition, i.e. when the inflatable support structure 14 including the ribs 16 is inflated. By comparing FIGS. 2 and 3 it can be seen that the inflatable support structure 14 comprises ribs 16 that are embedded or integrated into the cushion 12. The inflatable support structure 14 comprises circumvention ribs 16″ near the facial plane and near the connection interface 24 as well as in-between-ribs 16′″ connecting the circumvention ribs 16″ with each other. The in-between-ribs 16′″ are used to create a rigid structure, when the inflatable support structure 14 is inflated. The circumvention ribs 16″ and the in-between-ribs 16′″ may internally be connected with each other, i.e. form a common and integrally inflatable rib structure.

As it can be further seen from FIG. 3, silicone flaps 28a, b may be provided to improve the sealing function and skin interaction. The silicone flaps 28a, b may either be separate parts that are connected to the cushion 12 or integral parts of the cushion 12. It is to be noted that in the illustrated example the silicone flaps 28a, b actually form one common flap 28 that surrounds the cushion 12 at the face-to-mask interface. However, depending on the technical application the respiratory interface device 10 may also comprise more than one silicone flap 28.

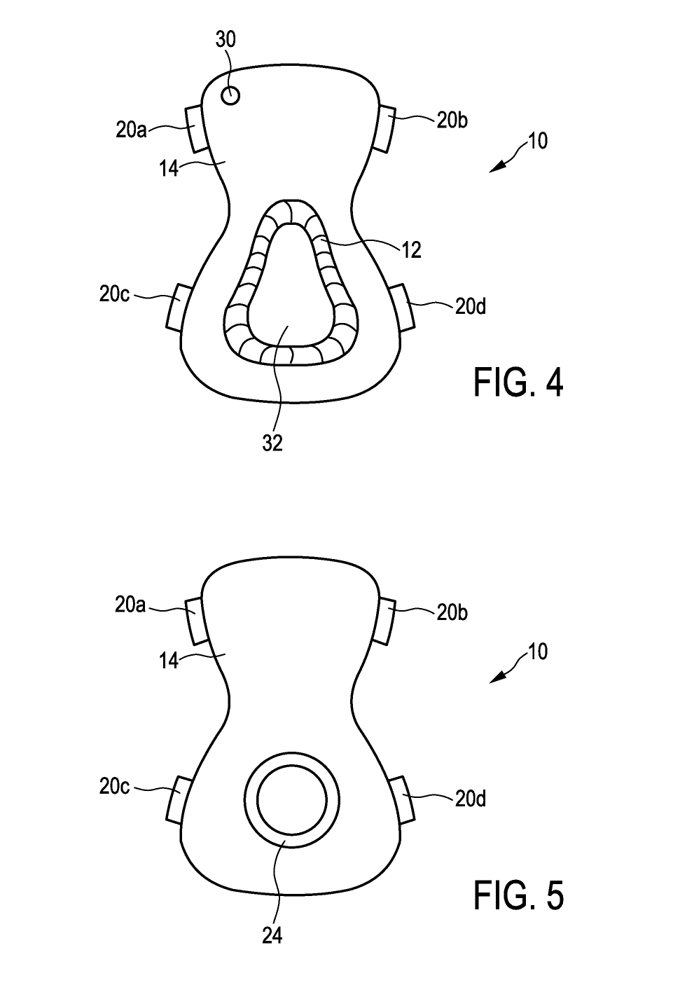

FIGS. 4 and 5 schematically illustrate a second embodiment of the respiratory interface device 10 according to the present invention shown from two different sides. In this embodiment the inflatable support structure 14 comprises a flexible, elastic foil. Preferably, the inflatable support structure 14 is made of a flexible, elastic foil. Heat sealing foils have shown suitable characteristics for this use. A relatively rigid foil has especially shown to be advantageous. However, the foil should not be to rigid, i.e. the foil should have also elastic properties, since a totally rigid foil is not sufficient to follow the facial contour. Elastic properties will enable this and distribute the pressure better over the contact area between the mask and the face of the patient.

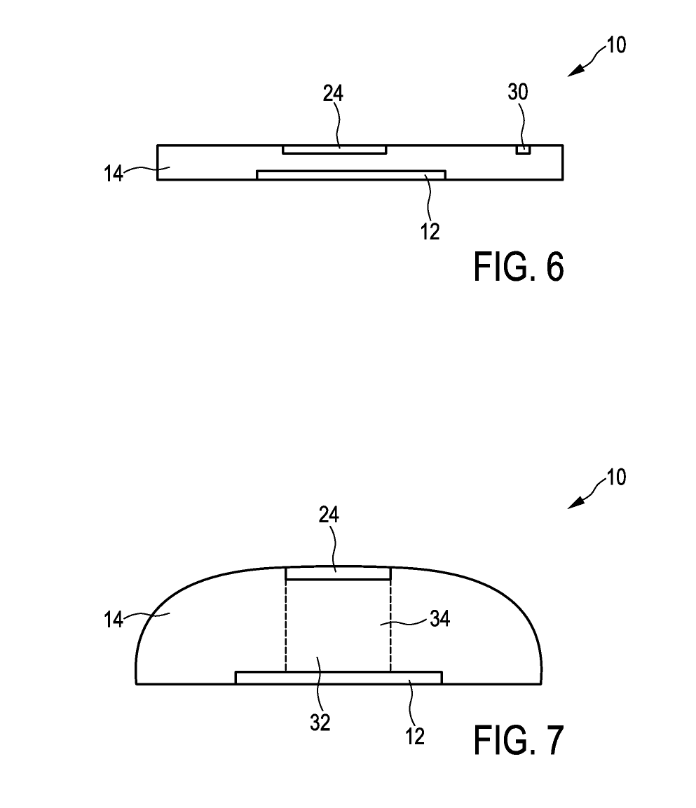

Instead of a rib-structure as shown according to the first embodiment (see FIGS. 2 and 3) this embodiment makes use of an inflatable foil that has substantially flat shape in the deflated condition, in particular a shape of a flat sheet. A flexible elastic foil enables a rigid smooth surface under proper tension, where rigid foils show wrinkling. The inflatable support structure 14 may, for example, be inflated by supplying air or another pressurized gas through a gas inlet 30 into the interior of the foil structure. As it can be seen by comparing FIGS. 6 and 7 the support structure 14 is thereby blown up, similar like an inflatable air mattress. FIG. 6 shows the deflated condition of the inflatable support structure 14, wherein the respiratory interface device is almost flat packed. FIG. 7 instead shows the inflated condition of the inflatable support structure 14, wherein the respiratory interface device is ready for use.

In the inflated condition of the support structure 14, the support structure 14 and/or the cushion 12 is preferably adapted to the shape of the patient's face. The cushion 12 in this case forms a cavity 32 for receiving the nose and/or mouth of the patient 50. In the shown example the cushion 12 has the shape of a ring that is adapted to the contours of the patient's face, i.e. adapted to the contours around the mouth, the cheeks and the area between the eyes (the nose bridge). The cavity 32 that is formed by the cushion 12 and serves for receiving the nose and/or mouth of the patient 50 is usually connected via a channel 34 with the opening 24 that serves as connection interface for connecting the air supplying hose to the respiratory interface device 10. This connection interface 24 is preferably arranged on the other side of the inflatable support structure 14, which is opposite to the side on which the cushion 12 is arranged.

The inflatable support structure 14 may in this case be realized by a single-layer, a double-layer or a multi-layer inflatable plastic foil. The provision of such an inflatable plastic foil in combination with a small size gas inlet 30 enables an easy inflation as well as an easy deflation of the support structure 14. The flexible plastic foil the inflatable support structure 14 is made of enables a very low-cost solution of the proposed respiratory interface device 10.

The cushion 12 may itself also be inflatable. However, it may also be made of silicone or comprise a gel pad as mentioned before. In case the cushion is inflatable it may either be inflated via a separate gas inlet (not shown) or internally connected with the inflatable support structure 14, so that it may be inflated via the same gas inlet 30. However, in a variant it is also possible that an interior part of the support structure 14 and/or the cushion 12 is connected to the connection interface 24, wherein the connection interface 24 is arranged to also inflate the support structure 14 and/or the cushion 12. In this case the inflation of the cushion 12 and/or the support structure 14 is combined with the gas/air supply to the airway of the patient 50. In other words, the inflation of the support structure 14 and/or the cushion 12 may either be realized by an additional pump filling the support structure 14 and/or the cushion 12 through a separate gas inlet 30 or by the available pressure that is supplied through the air supplying hose connected to the connection interface 24. It is also conceivable, that the air supply may be switched between two states, a first state, in which the air is supplied to the patient's airway, and a second state, in which the air is used to inflate the support structure 14 and/or the cushion 12.

Similar as the first embodiment shown in FIGS. 2 and 3, the second embodiment of the respiratory interface device 10 (FIGS. 4 to 7) may also comprise connection members 20a-d for attaching head gear straps (not shown) to the respiratory interface device 10. Similar as the support structure 14 and the cushion 12, these head gear straps may also be partly or fully inflatable.

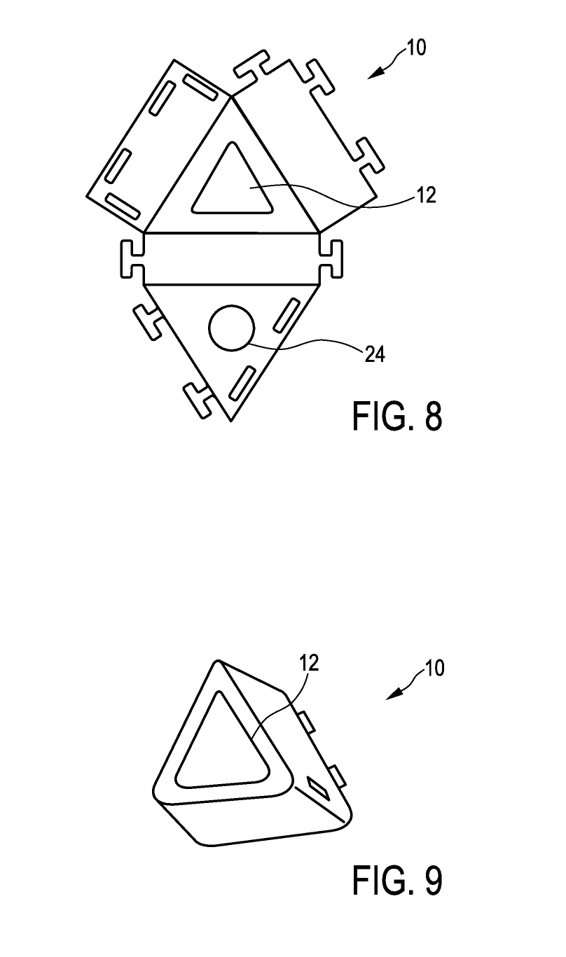

A third embodiment of the respiratory interface device 10 according to the present invention is shown in FIGS. 8 and 9. As shown therein, the respiratory interface device 10 is adapted to be foldable from an unfolded, substantially flat shape to a folded shape. FIGS. 8 and 9 only schematically visualize the principle of this embodiment.

In this embodiment, the cushion 12 and/or the support structure 14 and/or the whole respiratory interface device 10 (mask) may be foldable. In addition, each of these parts may be inflatable as explained above. The mask 10, the cushion 12 and/or the support structure 14 may thus be arranged to be assembled from an initially flat package. The mask 10, the cushion 12 and/or the support structure 14 may comprise or consist of a disposable material, such as paper or consumer grade plastics or other low cost materials. The patient interface 10 may then be manufactured from a flat sheet of material. By folding along predetermined lines and connecting predetermined edges, the patient interface 10 may be assembled from the initially flat sheet. Such an assembly from a flat package allows storage and transport of the respiratory interface device 10 while it is still a flat package.

Consequently, compared to bulky and rigid masks as known from the prior art, storage and transport become easier. In order to get the mask ready for use it only needs to be assembled by folding and then partly or fully inflated as explained above.

In summary, the inventors have found a way for a very lightweight and low-cost respiratory interface device that makes use of an air enforced structure. The proposed respiratory interface device is easy to handle, due to its lightweight comfortable to wear and easy and space-saving to store and transport. The proposed respiratory interface device is therefore optimal to be used on travel and in other situations where a space-saving and lightweight solution is of need.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A respiratory interface device for delivering a gas to a user, comprising:
a connection interface for connecting a hose to deliver gas to the user,
a cushion that is adapted to contact a face of the user and to surround at least a part of a nose and/or a mouth of the user, and
an inflatable support structure, for holding the cushion.

2. A respiratory interface device according to claim 1, wherein the inflatable support structure is adapted to form a mask shell for holding the cushion when the support structure is inflated.

3. A respiratory interface device according to claim 1, wherein the cushion is releasably connected to the inflatable support structure.

4. A respiratory interface device according to claim 1, wherein the cushion is embedded into the inflatable support structure.

5. A respiratory interface device according to claim 1, wherein the inflatable support structure comprises at least one inflatable rib that is adapted to form a frame for holding the cushion and for providing mechanical stability when the at least one rib is inflated.

6. A respiratory interface device according to claim 1, further comprising a rigid frame for providing mechanical stability to the respiratory interface device and holding the inflatable support structure and/or the cushion.

7. A respiratory interface device according to claim 1, wherein the support structure is inflatable from a deflated to an inflated condition, wherein the support structure has a substantially flat shape in the deflated condition.

8. A respiratory interface device according to claim 1, wherein the inflatable support structure and/or the cushion is at least partly made of an elastic foil.

9. A respiratory interface device according to claim 1, wherein the cushion is at least partly inflatable.

10. A respiratory interface device according to claim 1, further comprising a gas inlet for inflating the support structure.

11. A respiratory interface device according to claim 10, wherein the cushion is connected to the inflatable support structure and inflatable via the gas inlet.

12. A respiratory interface device according to claim 1, wherein an interior part of the support structure and/or the cushion is connected to the connection interface, and wherein the connection interface is arranged to inflate the support structure and/or the cushion.

13. A respiratory interface device according to claim 1, wherein the respiratory interface device comprises a headgear that is adapted to fixate the respiratory interface device to the user's head, wherein the headgear is at least partly inflatable.

14. A respiratory interface device according to claim 1, wherein the respiratory interface is adapted to be foldable from an unfolded, substantially flat shape to a folded shape.

15. An inflatable support structure for use in a respiratory interface device as claimed in claim 1, for holding a cushion that is adapted to contact a face of a user and to surround at least a part of a nose and/or a mouth of the user.

16. An inflatable support structure as claimed in claim 15, wherein the inflatable support structure is arranged to be magnetically connected to the cushion.

17. A cushion for use in a respiratory interface device, wherein the cushion is arranged to be magnetically coupled to the respiratory interface device.
